(19) United States
(12) Patent Application Publication
(10) Pub. No.: US 2007/0092556 A1
(43) Pub. Date: Apr. 26, 2007

(54) Anti-microbial material and method of making the same

(75) Inventors: Fred Michael Schecter, New York, NY (US); Edward Meyer Schecter, Kinnelon, NJ (US)

Correspondence Address: GOTTLIEB RACKMAN & REISMAN PC, 270 MADISON AVENUE, 8TH FLOOR, NEW YORK, NY, 100160601, US

(73) Assignee: SOMMERS PLASTIC PRODUCTS Co., Inc., Clifton, NJ (US)

(21) Appl. No.: 11/335,380

(22) Filed: Jan. 19, 2006

Related U.S. Application Data
(60) Provisional application No. 60/729,529, filed on Oct. 24, 2005.

(51) Int. Cl. A61K 33/38; A61K 9/70

(52) U.S. Cl. 424/443; 424/618

(57) ABSTRACT

A material to be used for furniture, drapery, wall covering and other similar objects in hospitals and the healthcare industry is formed of a skin with an inorganic anti-microbial compound attached to a backing. The skin includes a film impregnated with said compound and one or more layers of polyurethane foam. The backing may be a woven or non-woven fabric.

### RELATED APPLICATIONS

This application claims priority to provisional application Ser. No. 60/729,529 filed Oct. 24, 2005, incorporated herein by reference.

### BACKGROUND OF THE INVENTION

A. Field of Invention

The present invention relates generally to a material that can be used as upholstery for covering home or institutional furnishings, drapery and room dividers. More particularly, the present invention pertains to a sheet that contains inorganic anti-biological agents, such as zeolite with silver ions, that provide the sheet with anti-microbial, anti-mold and anti-fungal properties for an extended time period.

B. Description of the Prior Art

There is a growing interest today in many types of products with anti-mold, anti-microbial and anti-fungal properties. These products are in high demand in nosocomial areas such as hospitals, doctor-patient waiting rooms, nursing homes, hospices, hotels, and so on. In general, these products are very desirable in any area that is used indiscriminately and is in contact by a large number of people, such as patients and others who may be exposed to infections, e.g., staph infection, SARS (severe acute respiratory syndrome) and other infections which have poor antibiotic response. There have been a number of attempts to make such products. However, many of these attempts, particularly using organic anti-microbial agents, have not been ideal in preventing infections. A further problem is that many such organic anti-microbial materials are effective only on the surface of products and after a relatively short time period, they wear off.

Examples of some organic types of antimicrobial agents are disclosed in U.S. Pat. Nos. 5,408,022 and 5,494,987 (an anti-microbial polymerizable composition containing an ethylenically unsaturated monomer, a specific one-, di- or tri-functional anti-microbial monomer and a polymerization initiator which can yield an anti-microbial polymer from which the anti-microbial component is not released), U.S. Pat. No. 5,709,870 (a silver containing anti-microbial agent which comprises carboxymethylcellulose, a cross-linked compound, containing silver in the amount of 0.01 to 1% by weight and having a degree of substitution of carboxymethyl group of not less than 0.4 and the anti-microbial agent being a silver salt of carboxymethylcellulose, which is insoluble in water), U.S. Pat. No. 5,783,570 (an organic solvent-soluble mucopolysaccharide consisting of an ionic complex of at least one mucopolysaccharide and a quaternary phosphonium, an antibacterial antithrombogenic composition comprising organic solvent-soluble mucopolysaccharide and an organic polymer material, an antibacterial antithrombogenic composition comprising organic solvent-soluble mucopolysaccharide and an inorganic antibacterial agent, and to a medical material comprising organic solvent-soluble mucopolysaccharide).

Examples of some inorganic types of anti-microbial agents are: Copper, silver or zinc compounds and fine particles of aluminum, iron and zinc.

Japanese Patent No. 1246204 discloses an anti-microbial thermoplastic article with copper as a compound added to the melted polymer just before extruding, in which the anti-microbial material is said to be resistant to washing.

U.S. Pat. No. 5,180,585 discloses an antimicrobial with a first coating providing the antimicrobial properties and a second coating as a protective layer. A metal having antimicrobial properties is used including silver which is coated with a secondary protective layer.

Japanese Patent No. 2099606 discloses a fiber with anti-microbial properties made of a liquid polyesther and inorganic micro particles of zinc silicate, both being added to the melted polymer after polymerization and just before extrusion.

The use of anti-microbial agents in connection with thermoplastic material is disclosed in U.S. Pat. No. 4,624,679. This patent is concerned with the degradation of anti-microbial agents during processing. This patent states that thermoplastic compounds which are candidates for treatment with anti-microbial agents include material such as polyamides (nylon 6 or 6,6), polyvinyl, polyolefins, polyurethanes, polyethylene terephthalate, styrene-butadiene rubbers.Japanese Patent No. 2091009 and U.S. Pat. No. 5,047,448 disclose an anti-microbial thermoplastic polymer with copper or zinc compounds and fine particles of Al, Ag, Fe and Zn compounds and a liquid polyesther, in which the anti-microbial material is said to be resistant to washing.

Japanese Patent No. 2169740 discloses a thermoplastic fiber such as PET which uses silver, copper or zinc as an anti-microbial agent. There is a cellulose component which reduces the amount of thermoplastic with anti-microbial agent and reduces the cost.

Examples of inorganic types of silver anti-microbial agents incorporating zeolite are disclosed in U.S. Pat. Nos. 4,911,898, 5,094,847, 4,938,958 (use of zeolite with exchangeable ions such as silver and others), U.S. Pat. No. 5,244,667 (an anti-microbial composition which involves use of partial or complete substitution of ion-exchangeable metal ion such a silver, copper, zinc and others), U.S. Pat. No. 5,405,644 (an anti-microbial fiber having a silver containing inorganic microbiocide and the silver ion is stated to have been supported by zeolite, among other materials, the purpose being to prevent discoloration).

While these anti-microbial agents are designed to prevent the development of bacteria, including resistant strains, the use of metal-containing materials presents the added difficulty of properly dispersing the anti-microbial agents throughout the material. Since these metal-containing compounds have existed as fairly large size particles (10 microns and greater), the ability to evenly mix or distribute them is limited. In addition, because of this size problem, these substances must necessarily be applied to the surfaces of materials instead of being incorporated into them. The latter causes the additional disadvantage of making the applied anti-microbial agents vulnerable to washings and abrasion. The nature of this product in claim 1 avoids these difficulties by homogenously incorporating and encapsulating zeolites with silver nano-sized particles in the resin strictly within the outer layer of such material.

Other patents relate to anti-microbial materials being added to materials. For example, U.S. Pat. No. 3,959,556 (1976) relates to synthetic fibers that incorporate an anti-microbial agent. U.S. Pat. No. 4,624,679 (1986) mentioned above, uses anti-microbial agents in connection with thermoplastic materials. These materials are formed by mixing polyamide resins, anti-microbial agents, and an antioxidant for reducing the degradation of the anti-microbial agent at the high temperatures necessary for processing.

Several other patents describe anti-microbial materials in which the anti-microbial agent is resistant to being washed away. U.S. Pat. No. 4,919,998 (1990) discloses an anti-microbial material that retains its desirable properties after repeated washings.

However, these materials have two inherent commercial disadvantages. First, while the anti-microbial agents incorporated into them do show some resistance to repeated washing, these agents do leach out of the materials, primarily because they are not physically incorporated into them. In fact, in many cases, the anti-microbial agents are only loosely bound into the material and are relatively easily washed away and can be naturally abraded away over time. On the other hand, if the agents are buried too deeply in the material or not homogeneously distributed, they will not make adequate contact with microbes and the economics of usage will be adversely affected.

Secondly, the anti-microbial agents used in these applications are generally organic substances. The disadvantage of these agents when used as anti-microbial agents is that bacteria can develop a resistance to their action. Thus, one is faced with the emergence of bacterial strains that are no longer affected by these anti-microbial agents; so-called “super bugs,” which negate the function of these materials and present an environmental risk.

Institutional furnishings are subject to excessive wear and tear. These furnishings must withstand the constant onslaught of dirt and spills of a variety of substances. They must also stand up to frequent cleanings with industrial strength cleansers. As a result, these furnishing coverings could be made stronger and more resistant by properly using anti-microbial and anti-fungal agents in their manufacture. The limited prior art approaches of coating fibers and/or fabrics with anti-microbial or anti-fungal materials have had only limited success.

Home furnishings are not subjected to as much wear and tear as institutional furnishings and can be made of materials which have softer “feel” and are usually more delicate than those made for institutional use. Therefore, it is difficult to make such materials which will stand up to repeated washings and to wear, particularly when they have been prepared with additives for special properties such as anti-microbial agents.

U.S. Pat. No. 3,983,061 for a process for the permanent finishing of fiber materials, including carpets, discloses an aqueous acid liquid for finishing fiber materials especially dyed carpets to make them anti-static, dirt-repellent, and optionally anti-microbial using a single bath process for finishing dyed textile floor coverings to make provide these characteristics to them. It states that the properties are “permanent” and defines this to mean retaining the properties after a “prolonged” period of wear and tear. However, the anti-microbial properties are not believed to last sufficiently long to be of commercially useful application, and the anti-microbial agent disclosed is organic in nature.

U.S. Pat. No. 4,371,577 for an anti-microbial carpet containing amino acid type surfactant is incorporated into fibrous materials prior to or after fabrication into a carpet using an organic material. The fibrous materials can be polyamide acrylic, polyester or polypropylene fibers. The preparation is accomplished in two manners. The first is that the pile yarns, the carpet foundations or the yarns for carpet foundation are subjected to the impregnation treatment with a surfactant, and the other is that a carpet fabricated from fibrous materials is impregnated with an organic material.

U.S. Pat. No. 5,762,650 for a biocide plus surfactant for protecting carpets where the dyeing and anti-microbial finishing is performed simultaneously. The anti-microbial agent is an organic material.

While there are known anti-microbial agents, which claim to be designed to prevent the development of resistant bacterial strains, the use of metal-containing materials presents the added difficulty of being able to successfully disperse the anti-microbial agents throughout the fibers. Since these metal-containing compounds exist as fairly large size particles (10 microns and greater), the ability to evenly mix or distribute them is limited. In addition, because of this size problem, these substances must necessarily be applied to the fibers instead of being incorporated into them. The latter causes the additional disadvantage in that these agents could be washed off easily.

In other words, various materials, particularly organic antibiotics have been used in the past to provide anti-microbial and anti-fungal properties to plastic upholstery fabrics. One of the disadvantages of some of the prior art is that the anti-microbial additives are organic and many organic materials either act as antibiotics and the bacteria “learns” to defeat the compound, and, in addition, many of them can emit harmful dioxins. Also, many such additives are applied topically to the materials or fabrics and tend to wash off or wear off over time and become ineffective. A further disadvantage of using surface additives is that when the additives are washed off, they end up in the wastewater stream and contribute to water pollution.

Thus, there is an acute need for making or covering various products used in nosocomial areas with antimicrobial material that is highly effective, inexpensive, and long-lasting; one that does not wear off when washed or wiped, thus maintaining their potency as an integral part of the furniture into which they are incorporated.

### SUMMARY OF THE INVENTION

There are several inorganic compounds, such as silver and silver ions that generally have well known anti-bacteriological or anti-microbial properties. One advantage of such compounds is that germs cannot develop immunity against these compounds through overexposure. Commonly used antibiotics are organic and generally use only a single mechanism to control microorganisms. In comparison, inorganic antimicrobials kill organisms through multiple mechanisms, such as damaging cell walls, stopping cell multiplication and cutting off respiration.

Inorganic materials, such as zeolites, in the presence of moisture, pump out silver ions in exchange for sodium ions within the moisture. As can be seen from these examples, inorganic and organic antimicrobials work very differently, and any resistance to organic agents generally does not trigger a resistance to inorganic agents.

Sheet and upholstery materials used to cover furniture or other household products for various uses are vulnerable to the seeding and colonization of bacteria and fungi from various sources, thus exposing nosocomial areas to their uninhibited growth. For example, patient waiting rooms, hospital rooms, nursing homes and healthcare facilities are potentially highly contagious environments. This is especially dangerous to those with low or compromised resistance and lowered immunity. Thus, products made with these materials would benefit from having antibacterial and anti-fungal agents incorporated into them.

The present invention provides a multilayered polyurethane sheet for covering upholstery products. The sheet includes an outer layer in which the inorganic anti-microbial agents are efficacious, long lasting and greatly resistant to washing off or wearing off of the material into which it is incorporated. This product is beneficial in the reduction of harmful microbes on upholstered furniture particularly but not limited for use in healthcare environments, including areas of nosocomial infection and where SARS (severe acute respiratory syndrome) and other infections which have no or poor antibiotic response may be present.

The present invention also provides an anti-microbial polyurethane material combined with standard fibers for uses in anti-microbial finished upholstery fabrics that are able to withstand significant wear and washings and still maintain their effectiveness.

The invention provides a layered PU (polyurethane) film used in products that do not sustain and indeed reduces growth/propagation of bacteria adhered to the product in spite of other conditions conducive to survival and growth/propagation. Thus, the invention prevents odors, generation and growth of infected sites, as well as preventing clogging or coating or other self passivating phenomena.

The film provided herein can be combined with additional pigments for coloration and UV additives to withstand fading and degradation in upholstery fabrics exposed to significant UV light for the use in anti-microbial finished upholstery fabrics. Advantageously, other compounds may be added as well, including, fire retardants and other similar compounds.

Briefly, the film is a laminate that includes layers adhesively mounted on a substrate. The anti-microbial compounds are imbedded solely in the outer layer of the laminate so that, once it is installed as upholstery on a product, the anti-microbial compounds can take effect.

### BRIEF DESCRIPTION OF THE DRAWINGS

FIG. 1 shows a block diagram of a process for making a film with inorganic anti-microbial properties in accordance with the present invention;

FIG. 2 shows a flow chart for the process of FIG. 1; and

FIG. 3 shows a cross-sectional view of the final material obtained by the process of FIGS. 1 and 2.

### DETAILED DESCRIPTION OF THE INVENTION

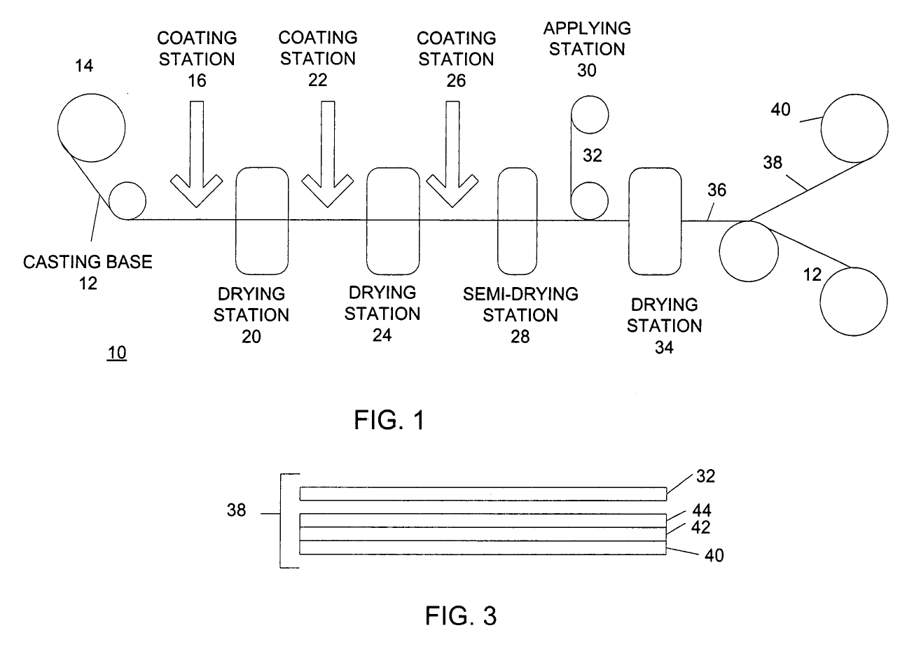

FIG. 1 shows an assembly line 10 for making a film in accordance with this invention. It should be understood that the line 10 is provided merely diagrammatically to illustrate the subject process and that the manufacturing process is somewhat more complicated.

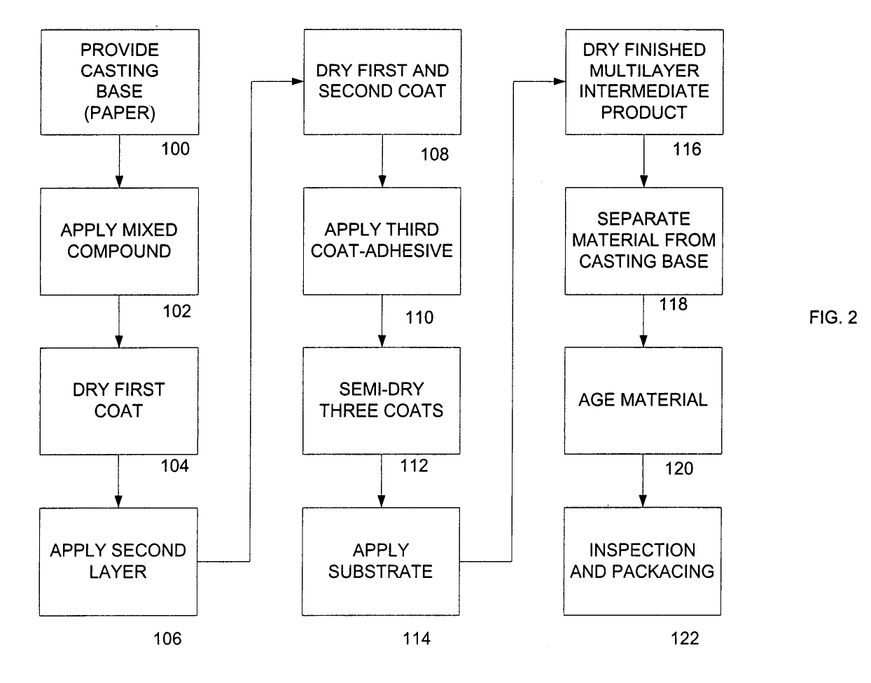

Referring to FIGS. 1 and 2, in step 100, first a casting base (such as paper) 12 is provided on a roll 14. This base 12 is fed to a first coating station 16 where a first coating of a mixed compound is applied (step 102). This mixed compound may be a thermoplastic polymer material such as a polyurethane resin including 3-5% silver ions. The silver ions can be provided in different forms. A preferred form is a silver zeolite. Other forms of silver may be used as well. For example, instead of silver ions, other inorganic antimicrobial materials may be used such as copper-, zinc-, or tin-based materials.

The resin is preferably a mixture of polyisocyanate, polyether polyol and polyesther polyol. Other polymer and non-polymer films may be used as well.

This mixture forms a PU (polyurethane) film on the base 12 having a thickness in the order of 0.002-0.003 in. This film is transported to a drying station 20 where it is dried at a temperature of 900-1300 degrees C. (step 104). Next, at station 22 a second layer is added (step 106). This second layer consists of a PU resin foam. This foam is well known in the prior art. The PU resin foam is dried at station 24 (step 108). Next, another coat of a PU resin is applied at station 26 (step 110). This later coat is only semi-dried at station 28 (step 112). As a result of this last process, the coat applied in at station 26 does not become solid but is somewhat soft and tacky. Next, at station 30 a polyester sheet or substrate 32 is pulled off from a roll 33 and applied on top of the last PU foam material (step 114). Sheet 32 is preferably a knit or woven material. Because the previous PU foam layer is soft and tacky, it readily adheres to the substrate 32. The resulting multilayer intermittent product is then dried at station 34 (step 116). The dried intermittent product 36 at this point is still supported on the base 12. After station 36 the paper 12 is separated from the final multi-layered material 38. The material 38 are then collected on a roll 40. The roll 40 is then allowed to age, preferably over 24-48 hours at a temperature of about 60-70 degrees C. (step 120). The process can occur at a relatively high speed, in the range of 7-14 meters/minute.

During the heating stages discussed above, the PU cures by a cross-linking process. This stage includes the mixing of the catalyst of an alphatic grade isocyanate prepolymer with an accelerant such as butylin laurate complex to cross-link polyurethane.

After aging, the material on drum 40 is inspected, packaged and shipped. Typically, the material 38, shown in cross-section in FIG. 3, includes a thin layer of inorganic anti-microbial agent (AgION) impregnated PU film 40 and two layers of PU foam 42, 44 deposited on the polyester fabric 32. Typically, the material 38 weight about 700 grams/sq. yd. and includes about 320 g/sq. yd (45.7%) of the fabric 32, 378 g/sq. yd. (54%) of PU resin and 2 g/sq. yd. (0.3%) of the inorganic anti-microbial agent (AgION).

Tests on material 38 indicate that the concentration of AgION silver ions in the film 40 can be in the range of 2-5% for the material to have effective anti-microbial properties.

The material 38 can be used for a variety of products normally found in hospitals, doctors' waiting rooms and other similar nosocomial areas. More specifically, the material may be used as upholstery for furniture such as chairs, tables, couches, and so on. The material may also be used for drapes window covering, wallpaper, room partitions, and so on.

Moreover, any furniture made or covered with this material is advantageous in that it resists wear and tear due to abrasion and it further has highly desirable anti-microbial properties. The cover material (product 38) may be further improved by adding to it other materials, including materials that make the product resistant to UV light, mold, fungus, etc.

To boost the effectiveness of the silver ions, the film may further include copper at a concentration of about 6.1% with 3.5% Ag ions.

Obviously, numerous modifications may be made to the invention without departing from its scope as defined the appended claims. For example, at one of the intermediate coating stations a foaming agent is added to prevent the PU from foaming. Alternatively, a separate station may be provided for applying this antifoaming agent.

I claim:

1. An anti-microbial material suitable for nosocomial areas, said material comprising:
an outer skin layer of thermoplastic polymer polyurethane (PU) resin impregnated with an inorganic anti-microbial compound; and
a backing supporting said outer skin layer.

2. The material of claim 1 wherein said outer skin layer contains an effective amount of inorganic anti-microbial additives in the PU outer skin layer that suppress substantially microbial growth and that inhibit fungal and mold activities.

3. The material of claim 1 wherein said anti-microbial compound is composed of silver ions.

4. The material of claim 3 wherein said silver ions are provided in a zeolite.

5. The material of claim 1 wherein said material is constructed to make at least one of upholstery, wall covering, office, room partitions, drapes and window treatments.

6. The material of claim 1, wherein the compound includes metallic ions selected from the group consisting of copper, zinc, tin and silver ions.

7. The material of claim 1, wherein said compound is one of a zeolite of silver, zirconium phosphate and dissolvable glass.

8. The material of claim 1, wherein the skin comprises approximately 2% to 5% zeolite with silver ions by weight.

9. The material of claim 1, wherein said skin includes an outer film of polyurethane and at least one layer of another material, said film including a zeolite of silver dispersed in a thermoplastic polymer polyurethane.

10. The material of claim 1, wherein said film further comprises a pigment that provides a uniform fade-resistant color.

11. The material of claim 1 wherein said skin comprises an outer film impregnated with said inorganic anti-microbial compound and a layer of polyurethane foam adhering to said fabric backing.

12. The material of claim 1 wherein said skin comprises an outer film impregnated with said inorganic anti-microbial compound, an intermediate layer of polyurethane foam and an inner layer of polyurethane foam, said inner layer adhering to said fabric backing.

13. A material for upholstery and other uses comprising:
A skin having an outer surface infused with an inorganic anti-microbial compound; and a backing adhesively attached to said skin.

14. The material of claim 13 wherein said skin includes a polyurethane film impregnated with said compound, an intermediate layer of polyurethane foam and an inner layer of polyurethane foam, said inner layer having adhesive properties for attachment to said backing.

15. The material of claim 13 wherein said backing is a fabric.

16. The material of claim 15 wherein said fabric is one of a polyester and a polyester/cotton blend.

17. The material of claim 13 wherein said compound contains metallic ions.

18. The material of claim 17 wherein said compound is selected from silver, tin, copper and zinc ions.

19. The material of claim 13 wherein said compound is silver zeolite.

20. The material of claim 13 wherein said film comprises about 2-5% of said compound by weight.
